(19) United States
(12) Patent Application Publication
(10) Pub. No.: US 2014/0323428 A1
(43) Pub. Date: Oct. 30, 2014

(54) ANTINEURITIC PHARMACEUTICAL COMBINATION AND COMPOSITIONS

(71) Applicant: LABORATORIOS SENOSIAIN S.A. DE C.V., Coloni Chapultepec Polanco (MX)

(72) Inventors: Hector Sensosiain Arroyo, Colonia Anahuac (MX); Maria Angelica Arzola Paniagua, Colonia Anahuac (MX)

(73) Assignee: LABORATORIOS SENOSIAIN S.A. DE C.V., Coloni Chapultepec Polanco (MX)

(21) Appl. No.: 14/365,988

(22) PCT Filed: Dec. 14, 2012
(86) PCT No.: PCT/IB2012/057345

§ 371 Date: Jun. 16, 2014

(30) Foreign Application Priority Data
Dec. 16, 2011 (MX) MX/A/2011/014042

(51) Int. Cl. A61K 31/55; A61K 31/51; A61K 31/714; A61K 31/197

(52) U.S. Cl. CPC A61K 31/55; A61K 31/197; A61K 31/51; A61K 31/714
USPC 514/52; 514/217

(57) ABSTRACT

The present invention refers to pharmaceutical combinations of two antiepileptic substances and also to combinations of an antiepileptic and B-complex vitamins, in which the antiepileptic substances are selected from pregabalin and oxcarbazepine, and the vitamins are selected from vitamin B1 and vitamin B12. The invention also relates to pharmaceutical compositions containing said combinations and to the use of said compositions for treating neuropathic pain (NP).

### FIELD OF THE INVENTION

The present invention relates to pharmaceutical combinations of two antiepileptic substances and combinations of an antiepileptic with B-complex vitamins, as well as pharmaceutical compositions containing said combinations, and the use of said compositions for the treatment of neuropathic pain (NP).

### BACKGROUND OF THE INVENTION

Pain includes a very complex and untransferable sensorial perception, which is difficult to describe, and even more difficult to evaluate or judge by an external person. To some extent, in a daily basis or in an isolated form, nobody has ever escaped to pain, provoking an organizational mind-body-spiritual disorder, to which the patient's conduct is added as expressed by the complaint, the high consumption of medicaments, irritability, displeasure, and loss of control of the own stress and thoughts.

Pain has been classified in several ways, according to its temporality or its intensity. According to its physiopathology, pain is classified as nociceptive, neuropathic and psychogenic.

Neuropathic pain is a disease of the nervous system that produces pain of varying intensity and can be from mild, moderate and up to severe. Neuropathic pain comprises a set of painful syndromes of varied aetiology, but which always include allodynia (pain produced by a stimulus that normally does not cause pain; sensitivity increase), hyperalgesia (higher pain sensation than the one commonly experimented after a nocive stimulus, and which is felt in an area that is more extensive than usual), spontaneous or evoked pain and dysesthesia (sensitivity reduction or exaggeration).

For pain treatment, different kinds of drugs are used, such as analgesics, non-steroidal antiinflammatory, opioids, antiepileptic substances, among others, however, for the treatment of pain having a physiopathological origin, specifically for neuropathic pain (NP), there is still a need for an efficient and effective pharmaceutical composition for attending this condition with fewer adverse effects.

The treatment of NP is currently attended with anti-depressive drugs such as amitriptyline, antiepileptic substances such as gabapentine, oxcarbazepine and pregabalin, or with noradrenaline inhibitors such as duloxetine.

The present invention offers a pharmaceutical composition containing the synergistic combination of antiepileptic and vitamins in a proportion such that it presents fewer adverse effects, due to the use of lower doses of the antiepileptic compound.

In the present invention a preferred embodiment of antiepileptic is pregabalin, a potent antiepileptic, a gamma-aminobutyric acid analog, indicated in peripheric and central neuropathic pain, epilepsy, generalized anxiety disorders and fibromyalgia. Another embodiment of the present invention employs oxcarbazepine, a carbamazepine derivative, which is also a potent antiepileptic and mood stabilizer, used mainly in the treatment epilepsy and bipolar disorder. Pregabalin or oxcarbazepine in the state of the art, may also be identified as antineuritics.

Pregabalin is quickly absorbed after an oral dose, it exhibits maximum plasma peaks at an hour and a half, with a 90% bioavailability. It does not link to plasmatic proteins, having a minimal metabolism; about 98% is excreted by the urinary route, with a half-life of elimination of 6.3 hours.

In the treatment of epilepsy and generalized anxiety disorders and neuropathic pain, the usual or known pregabalin dose is from 75 mg to 600 mg per day, divided into 2 to 3 times per day. In neuropathic pain it is recommended administering 150 mg at the beginning, increasing up to 300 mg in 3 to 7 days and after de 7th day, increasing up to 600 mg. For patients with diabetic neuropathy, a maximum of 300 mg is recommended.

Pregabalin has demonstrated to be a good option for the treatment of NP at doses of from 300 to 600 mg, unfortunately, a minority of patients has been favored because the treatment is abandoned due to the presence of adverse reactions such as: dizziness (27-46%), drowsiness (15-25%), hand tremor, among others.

The present invention considers the use of pregabalin in a dose from 5 mg to 600 mg per day, administered from 1 to 3 doses per day, depending on the clinical experience of the physician.

Oxcarbazepine is a carbamazepine derivative to which an extra oxygen atom is added in the dibezazepine ring. This change helps reducing the adverse effect to the liver. It has a melting point of 215.5° C. and has low solubility in water (308 mg/L at 25° C.)

Oxcarbazepine is quickly absorbed orally and does not interact with food. In monotherapy, its initial oral use is 600 mg per day in two doses with a maximum of, in some cases, 2400 mg; in children older than 6 years the dose is from 6 mg to 10 mg/Kg in two doses per day. It is recommended as a first and/or second line drug; as with other antiepileptic substances, the adverse effects increase with dose increase.

The present invention considers the use of oxcarbazepine at a dose from 100 mg to 2500 mg per day, and in a preferred embodiment, a dose from 100 mg to 1200 mg administered from 1 to 3 doses per day, depending on the clinical experience of the physician.

Another component of the present invention is the vitamin compound, which is selected from B-complex vitamin such as vitamin B12, vitamin B1 or combinations thereof.

Vitamin B12 is the common name of a group of related cobalt-containing compounds, usually known as “cobalamins”, of which cyanocobalamin, methylcobalamin and hydroxocobalamin are the main forms of clinical use. For the present project, methylcobalamin is excluded and in one embodiment cyanocobalamin or hydroxocobalamin is preferred.

Vitamin B12 helps forming nucleic acids; it contributes to the normal functioning of red blood cells, it helps maintaining nerve cells and combating neuropathic pain. It is capable of reducing tactile allodynia induced by the linkage of the spinal nerves L5 and L6. In the treatment of pernicious anemia and other anemias, the dose is from 250 mcg to 1000 mcg. In case of neurologic affectation, it may be administered by intramuscular route at doses of 1000 micrograms. For mild cases or those of preventive character, its ingestion from 50 mcg to 150 mcg is suggested.

For the present invention the use of vitamin B12 is considered at a dose of from 50 mcg to 1000 mcg per day, administered as 1 to 3 doses per day, depending on the clinical experience of the physician.

Vitamin B1 is part of a coenzyme that decomposes and assimilates carbohydrates. This vitamin comprises complexes such as thiamine, benfotiamine, acethiamine HCl, bisbentiamine, cocarboxylase, among others. The present project excludes the use of benfotiamine and in one embodiment thiamine is preferred, preferably thiamine mononitrate or hydrochlorhide.

Vitamin B1 is an essential component of the nucleic acids, DNA and RNA (gene carriers). It promotes appetite and normalizes the nervous system functions, therefore it is essential for maintaining the functional integrity of the nervous, cardiovascular and digestive systems. Vitamin B1 is indicated in conditions produced by a low thiamine level inflammation of the nerves outside the brain (peripheral neuritis). Thiamine is also used in digestive problems, including lack of appetite, ulcerative colitis and chronic diarrhea. Thiamine is also used in the treatment of AIDS and to strengthen immunologic system; in diabetic pain, heart diseases, alcoholism, aging; in a kind of cerebral damage called cerebelous syndrome, for buccal ulcers, vision problems such as cataracts and glaucoma, in dizziness caused by movement, and for improving sports performance.

Thiamine is preferably administered orally; in deficiency cases the usual oral dose is 10 mg to 50 mg per day, with a maximum of 300 mg in a single or divided dose. In the treatment of the Wernicke-Korsakoff syndrome, it is administered from 500 mg to 750 mg with other vitamins, three times a day for at least two days.

The present invention considers the use of vitamin B1 in a dose of from 15 mg to 750 mg per day, being administered from 1 to 3 doses per day, depending on the clinical experience of the physician.

In general, the treatment of neuropathic pain with the combination of an antiepileptic and B vitamins, or with the combination of two antiepileptic substances, represents a great advantage in those patients that require maintaining their psychomotor, cognoscitive and alert capacities (workers, elder persons, among others) and could also improve the performance of their activities by rationing it at least once a day. Nevertheless, the combined therapy could generate adverse effects when using more than one drug, therefore it is not obvious to think that the combination of an antiepileptic such as pregabalin or oxcarbazepine and B vitamins, or the combination of two antiepileptic substances, will be an alternative to pharmaceutical compositions for the treatment of NP.

The purpose of the present invention is to offer a pharmaceutical composition containing any of the following combinations: a) pregabalin or its pharmaceutically acceptable salts, and vitamin B12; b) pregabalin or its pharmaceutically acceptable salts, vitamin B12 and vitamin B1; c) oxcarbazepine or its pharmaceutically acceptable salts, and vitamins B12; d) oxcarbazepine or its pharmaceutically acceptable salts, vitamin B12 and vitamin B1; and e) oxcarbazepine and pregabalin or its pharmaceutically acceptable salts.

In another preferred embodiment a composition with pregabalin, oxcarbazepine and vitamins may be presented.

Such combinations are useful for treating neuropathic pain with less adverse effects, in which the antiepileptic dose is from 3 to 5 times less than the conventional therapy of from 150 to 600 mg per day of pregabalin and from 600 mg to 2400 mg per day of oxcarbazepine. These combinations attend NP in a synergistic way, without the risks of the adverse effects resulting from a high dose of pregabalin or oxcarbazepine.

No published documents were found in the state of the art, related with the combinations: a) pregabalin-vitamin B12; b) pregabalin-vitamin B12-vitamin B1; c) oxcarbazepine and vitamins B12; d) oxcarbazepine-vitamin B12-vitamin B1; and e) oxcarbazepine-pregabalin, for use in neuropathic pain.

Some relevant references and their differences with respect to the present invention are mentioned below.

Documents Medina-Santillán et al., Proc. West. Pharmacol. Soc., No. 47, (2004) pp. 109-112 and Medina-Santillán et al. Proc. West. Pharmacol. Soc. No. 47, (2004) pp. 76-79 refer to the use of gabapentine and a mixture of vitamins B1, B6 and B12 for treating neuropathic pain. Document Mixcoatl-Zecuatl, et al., Methods and Findings in Experimental and Clinical Pharmacology, Vol. 30, No. 6 (2008) pp. 431-441 mentions the use of gabapentine or carbamazepine in combination with benfotiamine or cyanocobalamin for treating neuropathic pain, demonstrating that an anti-allodynic interaction occurs.

In contrast with the aforementioned documents, the present invention involves other antiepileptic substances, such as, pregabalin or oxcarbazepine, combined together, or each one in combination with a) vitamin B12 or b) vitamin B12 and vitamin B1. None of the aforementioned documents describes or suggests a synergistic interaction in the anti-allodynic effect of the combinations a) pregabalin+vitamin B12; b) pregabalin+vitamin B12+vitamin B1; c) oxcarbazepine+vitamin B12; d) oxcarbazepine+vitamin B12+vitamin B1; and e) oxcarbazepine-pregabalin.

A medical note localized on the page “efisiotherapy.net” mentions the treatment of nerve pain with B-complex vitamins (B1, B6 and B12) and pregabalin. This reference lacks of scientific support and only refers to a suggestion for the treatment. It does not suggest or describe a synergistic effect between pregabalin+vitamin B12 or pregabalin+vitamin B1+vitamin B12.

A pharmaceutical product commercialized in India and Japan for the treatment of neuropathic pain, contains a composition with pregabalin and methylcobalamin.

Modified-release pharmaceutical compositions exist in India, having 75 mg, 150 mg and 300 mg of pregabalin and 750 mg of methylcobalamin, as well as immediate-release compositions with 75 mg, 150 mg and 300 mg of pregabalin and 500 mg, 750 mg and 1500 mg of methylcobalamin. In contrast, the present invention refers to the synergistic combinations between a) pregabalin+vitamin B12 with the exception of methylcobalamin, and b) pregabalin+vitamin B1+vitamin B12 with the exception of methylcobalamin and benfotiamine, wherein such combinations present less adverse effects and higher efficacy.

Another pharmaceutical product Nervijen from Jenburkt Laboratory (India) contains a composition that includes pregabalin, methylcobalamine, pyridoxine, folic acid and benfotiamine. In contrast, the present invention refers to the combinations: pregabalin+vitamin B12, and pregabalin+vitamin B12+vitamin B1 with the exception of benfotiamine, which have a synergistic activity against nerve pain.

Application WO2010/002517 from Accelerarated Care refers to a method for treating peripheral neuropathy; at no moment it foresees the simultaneous administration of pregabalin and cyanocobalamin. It refers to the use of a dispositive for providing an electrical stimulus and optionally co-administering a substance selected from pyridoxine, thiamine, vitamin B12, gabapentine, pregabalin, among others.

Application WO2009/132119 from Auspex Pharm., comprises the muscle relaxant metaxalone and its simultaneous administration with any of pregabalin, methylcobalamin or oxcarbazepine, in the treatment of musculoskeletal disorders. This document does not foresee the simultaneous administration of pregabalin and vitamin B12, or pregabalin and oxcarbazepine, or vitamin B12 and oxcarbazepine. Besides, the present invention does not include metaxalone.

Application WO2001/012155 from Lipocine Inc. refers to modified-release compositions and manufacturing methods, for improving the absorption of hydrophilic drugs such as pregabalin or cyanocobalamin. At no moment it foresees the simultaneous administration of the antiepileptic and the vitamins.

Application WO2009/046801 from Merck refers to a composition comprising a thiamine derivative known as benfotiamine combined with pregabalin, gabapentine, or carbamazepine, being gabapentine the preferred one. It shows a synergistic effect for the combination benfotiamine+pregabalin and benfotiamine-gabapentine. This document does not mention or suggest the simultaneous administration of pregabalin or oxcarbazepine with vitamin B1 or vitamin B12, with the exception of benfotiamine, nor the combination of the two antiepileptic substances.

Application WO2009/004082 from Inserm refers to the use of a substance selected from: taurine, a taurine precursor, a taurine metabolite, a taurine derivative, a taurine analog or any substance required for taurine biosynthesis, such as thiamine or cyanocobalamin among others, in the manufacture of a medicament useful for inhibiting the undesirable effects of a drug that induces to high levels of extracellular GABA or to the increase in the GABA receptor activation. This document does not contemplate the simultaneous administration of pregabalin-vitamin B12, oxcarbazepine-vitamin, pregabalin-vitamin B12-vitamin B1, oxcarbazepine-vitamin B12-vitamin B12, or pregabalin-oxcarbazepine.

Application WO2009/126931 from Xvasive Inc. describes a method for treating obsessions associated with opioid withdrawal, comprising the administration of preferably buprenorphine and a second drug selected from antipsycotic, antiepileptic, cannabinoid, among others, from which oxcarbazepine and pregabalin may be selected. Besides, it considers a method for treating bipolar disorders with the administration of buprenorphine and other drug such as vitamin B12, pregabalin or oxcarbazepine. This document does not contemplate the synergistic combinations of the present invention including oxcarbazepine and vitamin B12, or pregabalin and vitamin B12, or pregabalin-oxcarbazepine.

Application WO2004/091578 from Biodelivery Sciences describes a method comprising the introduction of a drug cargo moiety to a liposome in the presence of a solvent, wherein the drug cargo moiety is selected from vitamins (B1, B6, B12, among others), oxcarbazepine, among others. It does not explicitly disclose the combination of oxcarbazepine with B-complex vitamins or with pregabalin. It neither suggests a synergistic effect with the combinations of the present invention.

Application WO2008/104996 from Jubilant Organosys refers to a water dispersable compressed tablet and its manufacturing process. The tablet may contain a plurality of active agents among which oxcarbazepine and vitamins in general, are found. It does not explicitly disclose the combination of pregabalin or oxcarbazepine with B-complex vitamins. It neither explicitly discloses the combination of pregabalin and oxcarbazepine.

Application WO2005048979 from Torrent Pharm. describes a modified-release composition comprising microtablets. Among the plurality of active agents it may contain oxcarbazepine and vitamins in general. It does not explicitly disclose the combination of pregabalin or oxcarbazepine with B-complex vitamins. It neither explicitly disclose the pregabalin-oxcarbazepine combination.

US Application No. US2010/0087422 from Gosford Centre (Holdings) PTY LTD. refers to a method for treating attention deficit by using one or more antiepileptic substances, however, it does not explicitly disclose the combination of pregabalin and oxcarbazepine, neither suggests a synergistic interaction between active agents.

Document Luszczki, J. J. et al., Epilepsy Research Vol. 91 No. 2-3, (2010) pp. 166-175 describes the anticonvulsive effect of the interaction of pregabalin with lamotrigine, oxcarbazepine and topiramate. This study does not consider the anti-allodynic effect of the combination pregabalin-oxcarbazepine.

Document May T. W. et al., Ther Drug Monit, Vol. 29 No. 6, (2007), pp. 789-794 refers to a study for determining the serum concentration of pregabalin in combination with other antiepileptic substances such as oxcarbazepine. This study concludes that the co-medication has a small but significant effect in the serum concentrations of pregabalin. This document does not suggest synergistic interaction of the pregabalin-oxcarbazepine combination in its anti-allodynic effect.

### BRIEF DESCRIPTION OF THE FIGURES

FIG. 1. Time course of the anti-allodynic effect produced by the oral administration of oxcarbazepine to rats con neuropathic pain. The relation is 50% withdrawal threshold against time. It is noted that oxcarbazepine increased the withdrawal retirement in dose-dependent way, which was interpreted as anti-allodyinic effect.

FIG. 2. Time course of the anti-allodynic effect produced by the oral administration of pregabalin to rats having neuropathic pain. The relation is 50% withdrawal threshold against time. It is noted that pregabalin increased the withdrawal threshold in a dose-dependent way, which was interpreted as anti-allodyinic effect.

FIG. 3. Time course of the anti-allodyinic effect produced by the oral administration of vitamin B₁₂ (cyanocobalamin) to rats having neuropathic pain.

FIG. 4. Analgesic effect of oxcarbazepine (O) alone, combined with a vitamin B₁₂ (B₁₂) dose, and combined with a mixture of vitamin B₁ and B₁₂ (B₁/B₁₂) in rats subjected to L5/L6 spinal nerve ligation. It is observed that vitamin B₁₂ produces a modest increase in the analgesic effect of oxcarbazepine, whereas the combination of oxcarbazepine with vitamins B₁ and B₁₂ substantially increases the effect of the antiepileptic. The data of oxcarbazepine alone were obtained with the compound in the form of crystals, whereas the combination was carried out with the compound in the form of powder.

FIG. 5. Analgesic effect of pregabalin (P) alone, combined with a dose of vitamin B₁₂ (B₁₂) and combined with a mixture vitamins B₁ and B₁₂ (B₁/B₁₂) in rats subjected to the L5/L6 spinal nerve ligation. It is observed that vitamin B₁₂ produces a modest increase in the analgesic effect of pregabalin, whereas the combination of pregabalin with vitamins B₁ and B₁₂ substantially increases the antiepileptic effect.

FIG. 6. Analgesic effect of oxcarbazepine (O) alone, the mixture of vitamins B₁/B₁₂, and the combination of oxcarbazepine with the mixture of vitamins B₁/B₁₂ in rats subjected to L5/L6 spinal nerve ligation. It is observed that the combination of oxcarbazepine and vitamins B₁/B₁₂ potentiates the analgesic effect as compared with the individual effect of the drugs.

FIG. 7. Analgesic effect of pregabalin (P) alone, the mixture of vitamins B₁ and B₁₂ and the combination of pregabalin with the mixture of vitamins B₁/B₁₂ in rats subjected to the L5/L6 spinal nerve ligation. It is observed that the combination of pregabalin and vitamins B₁/B₁₂ potentiates the analgesic effect as compared to the individual effect of the drugs.

FIG. 8. Analgesic effect of oxcarbazepine (O) alone and combined with the mixture of vitamins B₁ and B₁₂ in rats subjected to the L5/L6 spinal nerve ligation. It is observed that the combination of oxcarbazepine and vitamins B₁/B₁₂ potentiates the analgesic effect, which is observed as a right shift in the dose-response curve. The effect is represented as the percentage of maximum possible effect (MPE).

FIG. 9. Analgesic effect of pregabalin (P) alone and combined with the mixture of vitamins B₁ and B₁₂ in rats subjected to the L5/L6 spinal nerve ligation. It is observed that the combination of pregabalin and vitamins B₁/B₁₂ potentiates the analgesic effect, which is observed as a right shift in the dose-response curve. The effect is represented as the percentage of the maximum possible effect (MPE).

FIG. 10. Isobologram that shows the synergistic interaction produced by the oral co-administration of oxcarbazepine and pregabalin to rats with neuropathic pain.

### SUMMARY OF THE INVENTION

The present invention exhibits a novel combination of an antiepileptic and vitamins for treating diseases related with neuropathic pain, in such a way that they act in a synergistic, fast and sustained manner, exhibiting also pharmaceutical compositions containing the combination of such active agents, and a kit of parts that includes such combination.

The present invention also refers to a novel synergistic combination of two antiepileptic substances for treating diseases related with neuropathic pain, as well as pharmaceutical compositions containing said combination, and a kit of parts that includes such combination.

An embodiment of the present invention consists in a pharmaceutical combination containing a) pregabalin or its pharmaceutically acceptable salts, and Vitamin B12 or b) pregabalin or its pharmaceutically acceptable salts, Vitamin B12 and Vitamin B1. In a preferred embodiment, vitamin B12 does not include methylcobalamin and vitamin B1 does not include benfotiamine. In a preferred way, vitamin B12 essentially consists in cyanocobalamin or its acceptable pharmaceutically salts and vitamin B1 essentially consists in thiamine or its pharmaceutically acceptable salts. The invention also refers to compositions containing said combination, and a kit of parts including such combination, wherein the use of lower doses of pregabalin as compared with the use of the standard dose improves the therapeutic effect, with fewer adverse effects.

Another embodiment of the present invention consists in a pharmaceutical combination containing a) oxcarbazepine or its pharmaceutically acceptable salts and Vitamin B12, or b) oxcarbazepine or its pharmaceutically acceptable salts, Vitamin B12 and Vitamin B1. In a preferred embodiment, vitamin B12 does not include methylcobalamin and vitamin B1 does not include benfotiamine. In a preferred way, vitamin B12 consists essentially in cyanocobalamin or its pharmaceutically acceptable salts and vitamin B1 consists essentially in thiamine or its pharmaceutically acceptable salts. The invention also refers to compositions containing said combination, and a kit of parts that includes such combination, wherein the use of lower doses of oxcarbazepine as compared to the use of standard doses improves the therapeutic effect, with fewer adverse effects.

Another preferred embodiment of the present invention consists in a pharmaceutical composition containing the combination of pregabalin and oxcarbazepine or its pharmaceutically acceptable salts, and compositions containing said combination, and a kit of parts that includes such combination, wherein the use of a lower dose of pregabalin or oxcarbazepine, as compared with the use of the customary dose, improves the therapeutic effect, with fewer adverse effects.

### Justification of the Invention

The treatment of NP has a diverse aetiology. Currently it is being treated with anti-depressants, antiepileptic substances or noradrenaline inhibitors and combinations thereof, however due to the presence of adverse effects, therapy is abandoned in many occasions.

Due to the aforementioned, it is not obvious to envisage as a first election therapy for NP the use of a pharmaceutical composition containing the combination of an antiepileptic such as pregabalin or oxcarbazepine, or its pharmaceutically acceptable salts, and vitamins. It is neither obvious to use the combination of these two antiepileptic substances for the treatment of NP. However, surprisingly, the present invention exhibits synergistic interactions with the two antiepileptic substances, and with the antiepileptic with vitamin B12, or with vitamin B12 and vitamin B1, in which the use of lower doses of pregabalin or oxcarbazepine, or their pharmaceutically acceptable salts, as compared to the use of standard doses currently used, improves the therapeutic effect with the possibility of fewer adverse effects, also providing for higher safety in the continuation of the treatment.

The combinations and compositions of the present invention offer the following advantages:
They provide for a synergistic therapeutic effect for treating and/or preventing disorders caused, mediated and/or propagated by lesions, disfunctions, compression and transitory perturbation of the neuronal system
Maintained long term efficacy
They are synergistic combinations with high safety profile, which allows their administration for a longer period of time
A low dose formulation of pregabalin or oxcarbazepine for reducing adverse effects without compromising the therapeutic effect when attending neuropathic pain.
Lower incidence of adverse reactions thereby promoting treatment adherence
Significant reduction of the possible adverse effects provoked by the treatment with pregabalin or oxcarbazepine, such as somnolence, fertility reduction, tiredness, nausea, altered mood, difficulty for concentrating or paying attention, weakness, blurred vision, extremity tremor (in the case of oxcarbazepine), among others.
By administering a low dose of the antiepileptic, the possibilities of causing hyperhomocysteinemia, which in turn is associated with cardiovascular damage, decrease.
There is no pharmacokinetic interaction among active agents.
The present invention is recommendable as a first option therapy in the treatment of neuropathic pain, due to its safety.

### Description of the Invention

In a preferred embodiment, the present invention refers to a combination of an antiepileptic and vitamins, a composition containing said combination and pharmaceutically acceptable excipients or vehicles, as well as a kit of parts including such combination. The combination of the present invention demonstrated to be useful and synergistic in the treatment of neuropathic pain.

In another preferred embodiment, the present invention refers to a combination of oxcarbazepine and pregabalin or their pharmaceutically acceptable salts. It also refers to a pharmaceutical composition containing said combination and pharmaceutically acceptable excipients or vehicles, as well as a kit of parts that includes such combination. This combination also demonstrated to be useful and synergistic in the treatment of neuropathic pain.

The anti-allodynic effect of uncombined, orally administered pregabalin, oxcarbazepine and cyanocobalamin was evaluated, and subsequently a study was carried out to determine the analgesic interaction with a) pregabalin and vitamin B12; b) pregabalin, vitamin B12 and vitamin B1; c) oxcarbazepine and vitamin B12; d) oxcarbazepine, vitamin B12 and vitamin B1; and e) oxcarbazepine-pregabalin, in the relief of nerve pain in the rat.

6-week-old female Wistar rats having a body weight of 140-160 g, were used. Each rat was studied only once and sacrified. The Guidelines for pain investigations in animals (Zimmermann, 1983) were used in all the experiments.

The rats were anesthesized via intraperitoneal route with a mixture of ketamine/xylazine (45 and 12 mg/kg, respectively). A partial incision was carried out in the left transverse lumbar area for isolating and ligating the L5 and L6 left spinal nerves with a silk suture 6-0. The ligation was carried out next to the formation of the sciatic nerve and distal to the dorsal root ganglion. Subsequently, the wound was sutured. Conduct tests were performed 12 days after surgery.

For the determination of tactile allodynia, the rats were placed in individual plastic boxes having a stainless steel metallic mesh bottom during 30 minutes, for their adaptation. The tactile threshold test is based in the induction of the animal's left paw withdrawal to gentle mechanical stimuli. The paw was stimulated by applying mechanical force using different Von Frey filaments ranging from 2.36 g to 6.65 g. The thinner the filament's thickness is, the fewer force is applied, and viceversa. For stimulating the animal's paw, the intermediate filament (3.41 g) was firstly used. The left paw elevation in ten seconds was taken as a positive response and a stimulus with a lower caliber filament was applied. If no withdrawal of the animal's paw occurred, it was considered as a negative response, then a stronger stimulus was applied with the next filament until the animal responded.

Once a change in the response took place, either positive or negative, the stimulation was carried out 4 more times, taking a series of 6 patterns of positive and negative responses (Chaplan et al., 1994). The response patterns were tabulated and the 50% response threshold was calculated by using the Dixon formula (1980).

Graphs of the 50% withdrawal threshold (g) as a function of time (h) were constructed from the obtained data

50% withdrawal threshold (g)=(10^{[Xf+Kδ]})/10,000

wherein:

Xf: is the value of the last von Frey filament used (log units)

K: is the correction factor based on the tabulated value of positive and negative responses

δ: is the average difference between stimuli (log units)

From the data of the 50% withdrawal threshold, bar graphs and dose response curves were constructed for the percentage of anti-allodynic effect represented as the percentage of the maximum possible effect (% MPE), by using the following formula:

% MPE=[(AUC drug−AUC vehicle/(AUC sham−AUC vehicle)]×100

In order to demonstrate a possible analgesic potentiation with the two antiepileptic substances in combination with vitamins B₁ and B₁₂, dose-response curves were plotted for each individual agent and for their combination with the B₁/B₁₂ vitamin mixture (in a fixed dose of 300 and 3 mg/kg, respectively). In both curves the value of the ED₅₀ was determined. The shifting of the dose-response curve to the left and the increase in the analgesic effect with the vitamins, with respect to the individual antiepileptic substances, were considered as indicative of analgesic potentiation.

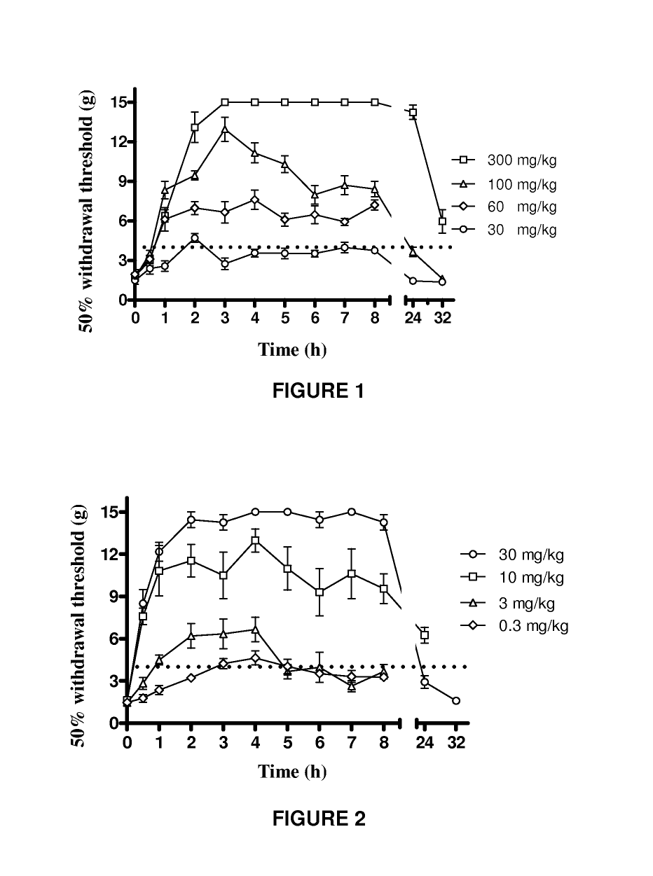

Oral administration of oxcarbazepine (30-300 mg/kg) significantly increased the withdrawal threshold, which was interpreted as an anti-allodynic effect (FIG. 1). The maximum anti-allodynic analgesic effect was observed at about 3 hours and decreased gradually at 8 hours. However, with the higher dose (300 mg/kg) the effect was maintained even for 24 hours. Motor incoordination starting 2 hours after the administration, and which was maintained up to 8 hours, was observed at this dose.

Oral administration of pregabalin (0.3-30 mg/kg) significantly increased the withdrawal threshold, which was interpreted as an anti-allodynic effect (FIG. 2). The maximum anti-allodynic analgesic effect was observed at about 2 hours and gradually decreased at 8 hours. At 24 hours the analgesic effect was no longer observed. With the higher dose (30 mg/kg) motor incoordination was observed between 1 and 7 hours, whereas with the 10 mg/kg dose, incoordination was observed between 3 and 4 hours after the administration.

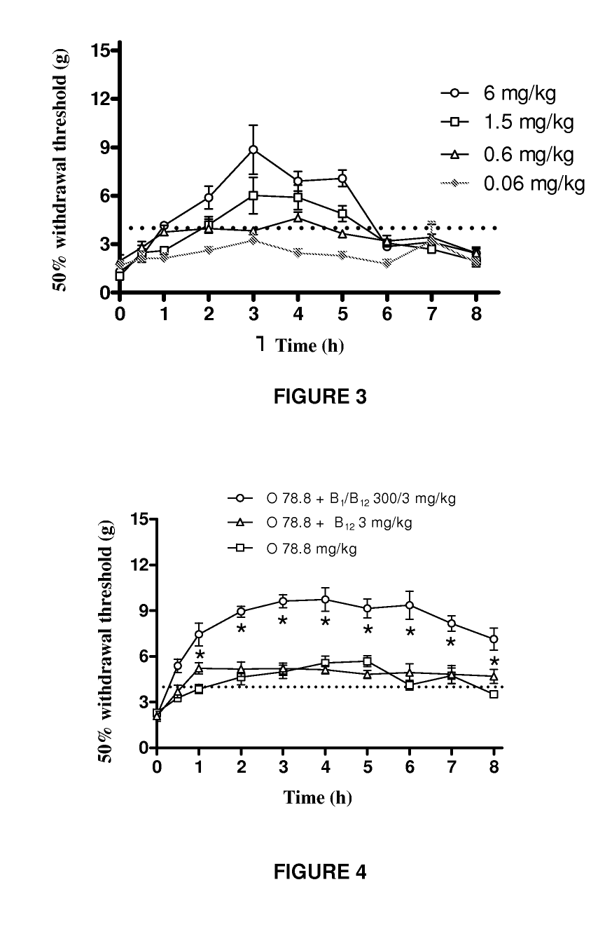

Oral administration of vitamin B₁₂ (0.06-6 mg/kg) significantly increased the withdrawal threshold, which was interpreted as an anti-allodynic effect (FIG. 3). The maximum anti-allodynic effect was observed at about 3 hours and decreased gradually at 6 hours. At 8 hours the analgesic effect was no longer observed. It is noted that vitamin B₁₂ increased the withdrawal threshold in a dose-dependent manner, which was interpreted as an anti-allodynic effect.

With these curves the ED₅₀ of oxcarbazepine and pregabalin, which were 78.8 and 3.3 mg/kg, respectively, were calculated.

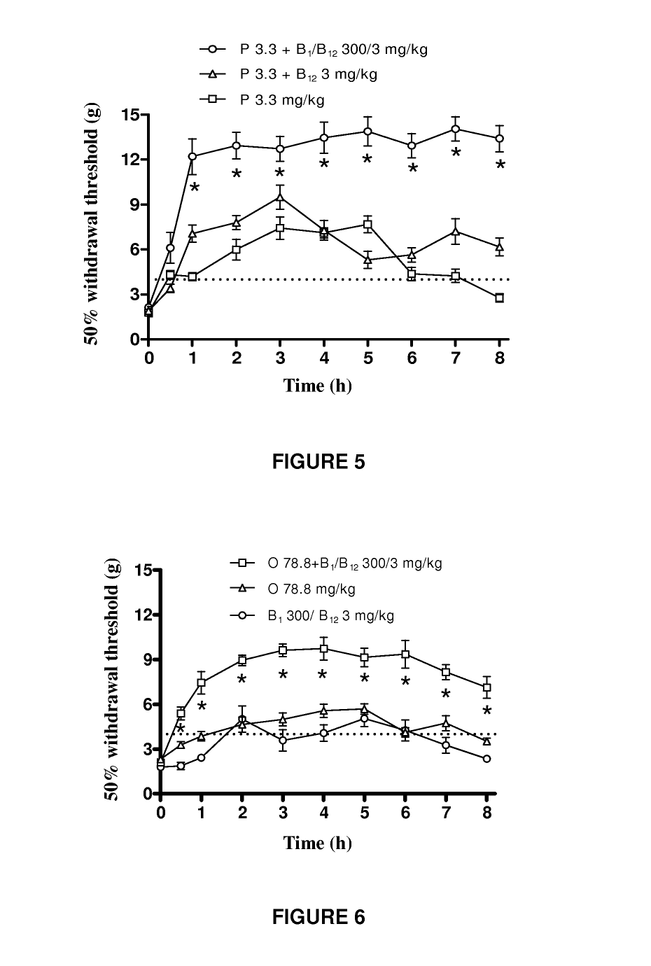

As a first step for trying to demonstrate an analgesic potentiation, the ED₅₀ of the antiepileptic substances alone, the vitamin B₁₂, or the combination of vitamins B₁ and B₁₂, was administered. The administration of vitamin B₁₂ produced a very small increase in the effect of the antiepileptic substances. In stark contrast, the mixture of vitamins B₁ and B₁₂ significantly increased the effect of oxcarbazepine or pregabalin (FIGS. 4 and 5). These data lead to the decision of using the mixture of vitamins B₁ and B₁₂ in further studies.

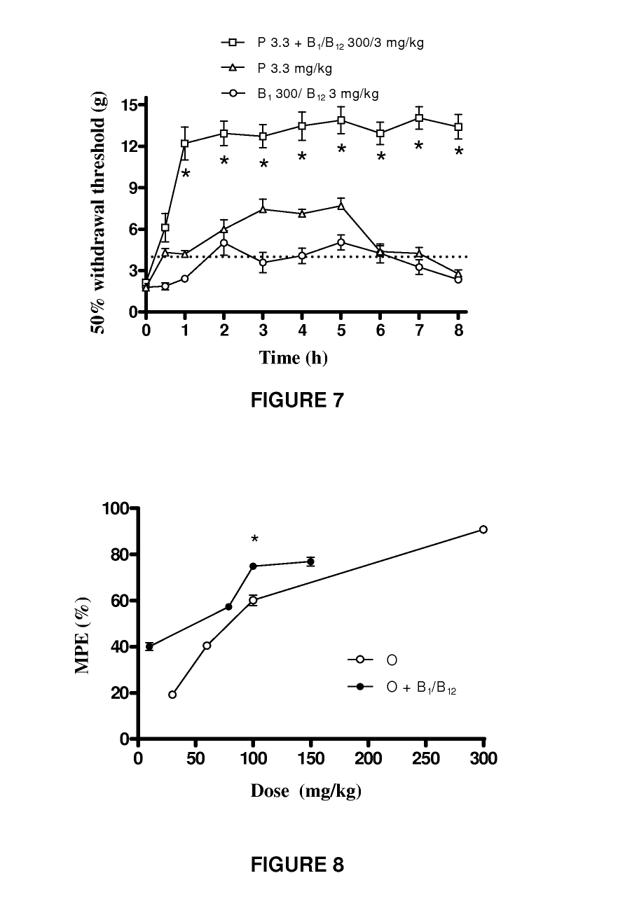

The administration of the mixture of vitamins (300 mg/kg of B₁ and 3 mg/kg of B₁₂) produced a lower anti-allodynic effect than the one obtained with the ED₅₀ of oxcarbazepine (FIG. 6) or pregabalin (FIG. 7). In contrast, the mixture of vitamins B₁ and B₁₂ significantly increased the effect of oxcarbazepine or pregabalin (FIGS. 6 and 7).

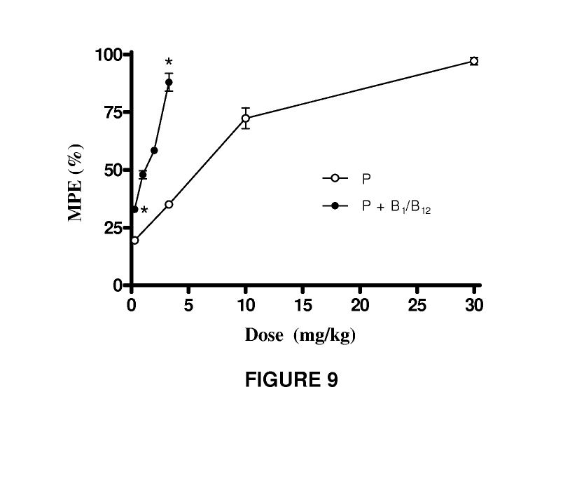

Following these curves, the dose-response curves of the two antiepileptic substances were carried out in the presence of a fixed dose of the vitamin mixture. The used doses of oxcarbazepine were 10, 78.8, 100 and 150 mg/kg whereas pregabalin doses were 0.3, 1, 2 and 3.3 mg/kg. In both cases they were combined with a fixed dose of the vitamin mixture (300 mg/kg of B₁ and 3 mg/kg of B₁₂). With both antiepileptic substances, a shifting to the left was observed in the dose-response curve (FIGS. 8 and 9). The ED₅₀ of the combinations were lower (51.87+9.8 and 0.85+0.2 mg/kg) than those of the antiepileptic substances alone (78.8 and 3.3 mg/kg for oxcarbazepine and pregabalin, respectively). Besides, an increase in the analgesic effect was observed in both cases. The best potentiation took place with the combination of pregabalin and the mixture of vitamins B₁ and B₁₂.

These data suggest that the combination of oxcarbazepine or pregabalin and the mixture of vitamins B₁ and B₁₂ produce an analgesic potentiation. This means that significantly lower doses are required for achieving the same level of the effect, which could importantly reduce the adverse effects of these two antiepileptic substances. Particularly, the combination of pregabalin and vitamins resulted as the most effective for alleviating neuropathic pain in the rat. Since the L5/L6 spinal nerve ligation model is predictive of what happens in the human being, the data suggest that these two combinations could be useful in the treatment of neuropathic pain in the human being.

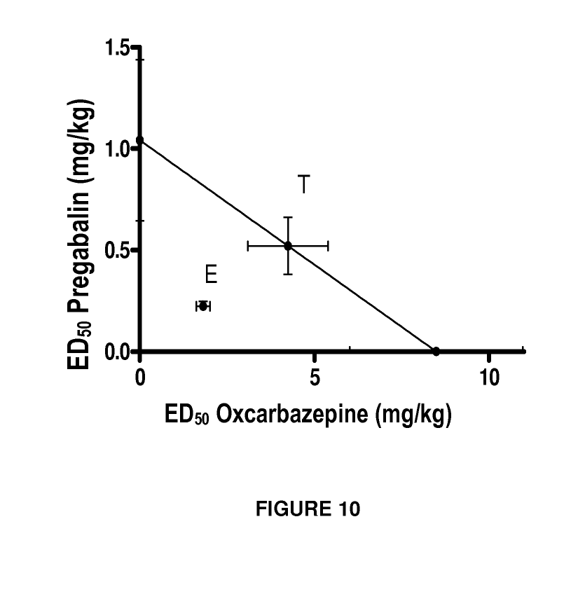

FIG. 10 shows that the interaction point between oxcarbazepine and pregabalin, is below the isobole line or additivity line, which indicates the existence of a synergistic effect; the isobole line is constructed from the representation of the effective doses of oxcarbazepine and pregabalin in each one of the graph axes.

The above data indicate and show that the novel combinations of a) pregabalin and vitamin B₁₂; b) pregabalin and vitamin B₁₂ and B₁; c) oxcarbazepine and vitamin B₁₂; oxcarbazepine and vitamin B₁₂ and B₁; and e) oxcarbazepine-pregabalin, produce a synergistic effect and are useful in the treatment of neuropathic pain (analgesic synergy). This means that lower doses are required, which could importantly reduce the adverse effects of these two anti-convulsivants.

Formulation of Pharmaceutical Compositions

The present invention encompasses the manufacture and use of pharmaceutical compositions comprising the following combinations: a) pregabalin and vitamin B₁₂; b) pregabalin and vitamin B₁₂ and B₁; c) oxcarbazepine and vitamin B₁₂; oxcarbazepine and vitamin B₁₂ and B₁; and e) oxcarbazepine-pregabalin; with one or more pharmaceutically acceptable excipients. These compositions may be presented in a form for oral, enteral, parenteral, topical, buccal, intranasal, ophthalmological, intrathecal administration, or another administration route. Controlled or sustained release formulations can also be prepared.

A formulation of a pharmaceutical composition of the present invention may be prepared in the form of tablet, solution, suspension, powder, capsules, granules, microsphere, microcapsules, emulsion or other spherical or non-spherical particle systems.

The tablets can be prepared by moulding or compressing the active agents with binding agents, lubricants, granulating agents, surfactants, disintegrants, diluents, and other excipients. Starch, sodium starch glycolate, etc. may be used as dispersing agents. Sodium lauryl sulfate may be used as surfactant, for example. Carbonates, lactose, microcrystalline cellulose, calcium phosphate, sodium phosphate may be used as diluents. Starch and alginic acid may be used as granulating agents and disintegrants. Gelatin, pregelatinized starch, polyvinyl pirrolidone, HPMC and HPC may be used as binding agents, for example. Magnesium stearate, stearic acid, talc, etc. may be used as lubricants. The tablets may or may not be coated and may further include sweeteners, flavoring agents, colorants, preservatives, etc.

The capsules containing the combination of the present invention may be soft or hard capsules, such as gelatin capsules, and may have a solid diluent such as calcium carbonate, sodium phosphate or kaolin, or an oily medium such as oil or liquid paraffin.

Liquid formulations in the form of solution and suspension may be prepared with aqueous or non-aqueous vehicles. Water and saline isotonic solution may be employed as aqueous vehicles. Vegetable oils, oily esters, ethyl alcohol, liquid paraffin, mineral oils, etc. may be used as non-aqueous vehicles. Suspensions also may include emulsifying agents, dispersing agents, moisturizers, preservatives, salts, flavoring agents, buffers, colorants, etc.

Powder or granule formulations may be adapted for direct administration to the patient, or in the form of tablets, or as a filling for capsules, or for preparing a suspension.

Emulsion formulations may be prepared by using vegetable oil, mineral oil or a combination thereof to form the oily phase. It may also include emulsifying agents such as acacia gum or tragacanth gum and esters, as well as sweeteners and flavoring agents.

The formulations of the present invention may also be adapted for rectal administration, for example, as suppositories or solutions for irrigation.

The formulations containing the present pharmaceutical combination may also be adapted for parenteral administration, such as inyectable solutions or suspensions, using solvents or diluents such as water, 1,3-butanodiol, sodium chloride solution.

Suitable formulations for topical administration may include liquid preparations, emulsions, solutions or suspensions.

The present pharmaceutical combination may also be formulated in a case or kit of parts in the form of two or three separate units of the components, i.e., the active agents exist in different pharmaceutical forms, for example, an active agent is found in a first pharmaceutical form (capsule, tablet, solution, suspension, granules, emulsion, powder, systems of particles or microparticles, etc.) and the other active agent is in a second pharmaceutical form (capsule, tablet, solution, suspension, granules, emulsion, powder, system of particles or microparticles, etc.).

Doses

For the combinations of anticonvulsivant with vitamins, pregabalin or its pharmaceutically acceptable salts, is found in a range from 5 mg to 600 mg, cyanocobalamin is found in a range from 0.1 mg to 10 mg, thiamine is found in a range from mg to 250 mg and oxcarbazepine or its pharmaceutically acceptable salts is found in a range from 600 mg to 2400 mg.

For the combinations of pregabalin and oxcarbazepine, pharmaceutically acceptable salts thereof, pregabalin is found in a range from 5 to 600 mg and oxcarbazepine in a range from 600 to 2400 mg.

Thanks to the efficacy of the synergistic combinations, doses of even a third or fifth part more reduced than the common doses, can be used.

### EXAMPLES

The invention will be described below with reference to the following examples, which are provided only for illustrative purposes.

### Example 1

A mixture of 150 mg pregabalin, 1 mg cyanocobalamin and mg thiamine mononitrate, was mixed with magnesium stearate, croscarmellose sodium, dibasic calcium phosphate, microcrystalline cellulose, HPMC, titanium dioxide and lactose. The mixture was placed in a capsule.

### Example 2

A mixture of 300 mg pregabalin, 1 mg cyanocobalamin and mg thiamine mononitrate, was mixed with magnesium stearate, dibasic calcium phosphate, microcrystalline cellulose, HPMC and lactose. The mixture was placed in a capsule.

### Example 3

A mixture of 300 mg oxcarbazepine, 1 mg cyanocobalamin and 50 mg thiamine hydrochloride was mixed with magnesium stearate, croscarmellose sodium, dibasic calcium phosphate, microcrystalline cellulose, HPMC, titanium dioxide and lactose. The mixture was placed in a capsule.

### Example 4

A mixture of 600 mg oxcarbazepine and 1 mg cyanocobalamin was combined with talc and alcohol and was granulated by solvent drying. The obtained granules were compressed to form tablets or were used to fill capsules.

The invention has been sufficiently described so that a person of ordinary skill in the art could reproduce and obtain the results mentioned in the present description. However, any person in the field of art related to the present invention may be able to make modifications not described in the present application. Therefore, if the application of such modifications in a determined composition requires the subject matter claimed in the following claims, such compositions shall be within the scope of the present invention.

1. A pharmaceutical combination for treating and/or preventing pain from moderate to severe and neuralgias of diverse location, characterized by comprising:
a) an antiepileptic selected from pregabalin or oxcarbazepine or its pharmaceutically acceptable salts; and
b) vitamins selected from: i) vitamin B12 or ii) vitamin B1 and B12, with the exception of methylcobalamine and benfotiamine.

2. The pharmaceutical combination according to claim 1, wherein vitamin B1 comprises thiamine hydrochloride or mononitrate and vitamin B12 comprises cyanocobalamin or hydroxocobalamin.

3. A pharmaceutical composition containing the pharmaceutical combination of claim 1.

4. (canceled)

5. The pharmaceutical composition in accordance with claim 3, in the form of suspension, tablet, granulate, powder, emulsion, solution, capsule, system of particles and microparticles.

6. (canceled)

7. The pharmaceutical composition in accordance with claim 3, wherein pregabalin is within a range from 5 mg to 600 mg, and oxcarbazepine is within a range from 100 mg to 2500 mg.

8. (canceled)

9. (canceled)

10. (canceled)

11. A kit of parts comprising the combination of claim 1, in the form of two or three separate units of the components.

12. A pharmaceutical combination characterized by comprising pregabalin and oxcarbazepine or its pharmaceutically acceptable salts, for treating and/or preventing pain from moderate to severe and neuralgias of diverse location.

13. A pharmaceutical composition according to the combination of claim 12 and pharmaceutically acceptable vehicles or excipients.

14. A pharmaceutical composition according to claim 13, wherein pregabalin or its pharmaceutically acceptable salts is within a range from 5 mg to 600 mg and oxcarbazepine or its pharmaceutically acceptable salts is found within a range from 100 mg to 2500 mg.

15. The pharmaceutical composition according to claim 13, wherein the composition is in the form of suspension, tablet, emulsion, granulate, powder, capsule, system of particles or microparticles.

16. (canceled)

17. A kit of parts comprising the combination of claim 12, in the form of two or three separate units of the components.

18. (canceled)

19. (canceled)

20. A pharmaceutical combination of claim 12, further characterized by comprising vitamins.

21. A method for treating and/or preventing pain from moderate to severe and neuralgias of diverse location, comprising administering the pharmaceutical composition of claim 3 to a patient in need thereof.

22. The kit of parts of claim 11, wherein the anticonvulsivant is found in a first pharmaceutical form selected from capsule, tablet, solution, suspension, powder, granules, emulsion and system of particles and microparticles, and the vitamin component is found in a second pharmaceutical form selected from capsule, tablet, solution, suspension, powder, granules, emulsion and system of particles or microparticles.

23. The kit of parts according to claim 14, wherein pregabalin is in a first pharmaceutical form selected from capsule, tablet, solution, granules, emulsion, suspension, powder and system of particles and microparticles and oxcarbazepine is in a second pharmaceutical form selected from capsule, tablet, solution, granules, emulsion, suspension, powder, and system of particles or microparticles.

24. A method for treating and/or preventing pain from moderate to severe and neuralgias of diverse location, comprising administering the pharmaceutical composition of claim 13 to a patient in need thereof.
